(19) United States
(12) Patent Application Publication
(10) Pub. No.: US 2004/0136912 A1
(43) Pub. Date: Jul. 15, 2004

(54) Safer toy balloons and methods for imparting unpalatable taste to same

(76) Inventors: Robert H. Murray, Fairport, NY (US); John R. Murray, Fairport, NY (US)

Correspondence Address: Robert H. Murray,, 52 Manor Hill Drive, Fairport, NY, 14450, US

(21) Appl. No.: 10/724,802

(22) Filed: Dec. 1, 2003

Related U.S. Application Data
(60) Provisional application No. 60/431,116, filed on Dec. 5, 2002.

(51) Int. Cl. A61K 49/00; B05D 3/00

(52) U.S. Cl. 424/10.4; 427/2.1

(57) ABSTRACT

A safe toy balloon and a method for making same are provided. The safe toy balloon includes an elastomeric inflatable member having an exterior surface, an interior surface, a neck portion and a body portion; and a safety coating formed on portions of the one or more of the surfaces of the inflatable member, the safety coating consisting essentially of repulsive taste agent for making uninflated and inflated balloons safer from risk of ingestion injuries or possible death from asphyxiation. The method of producing a safe toy balloon including (a) providing a dipping assembly containing at least one toy balloon material supporting mandrel; (b) providing a bank of vats, including at least one vat containing liquid toy balloon material; (c) coating the at least one toy balloon material supporting mandrel with a repulsive taste agent to achieve a repulsive taste coated mandrel; and (d) dipping the repulsive taste coated mandrel into the at least one vat containing liquid toy balloon material to form a toy balloon having a repulsive interior coating from the repulsive taste coated mandrel.

This Application is based on a Provisional Patent Application No. 60/431,116 filed Dec. 5, 2002.

### RELATED APPLICATIONS

This application is related to U.S. application Ser. No. ______ (Applicant's Docket MSD01, entitled “A SAFE TOY BALLOON CLOSURE AND SEALING DEVICE AND ASSEMBLIES USING SAME having a common inventor and filed herewith on the same day.

### FIELD OF THE INVENTION

This invention relates to toy balloons in general and particularly to elastometrc toy balloons and to methods for making uninflated and inflated balloons safer from risk of ingestion injuries.

### BACKGROUND OF THE INVENTION

For a number of decades, toy balloons have been the leading cause of death from toys in young children. According to the U.S. Consumer Product Safety Commission data, in a 10-year period ending October 2002 there were 81 deaths, 49 were under age three, 29 were ages three and over, with an average age of four years. An additional 118 incidents of injury involving toy balloons required medical treatment and were the patient was released on the same day. Ten incidents required further hospitalization.

It is common knowledge that young children, especially those three years of age and under, are inclined to place objects they find into their mouth. If the objects are small, sharp or breakable there is the possibility for injury by choking or even death by asphyxiation. There is a standardized choke test for children of all ages. The test apparatus for this choke test consists of a vertical tube 1 1/4 inch in diameter and 2-¼ deep with a partition extending upward at a 45-degree angle from a bottom corner of the tube. A small part, placed within the tube, is considered suitable for children of all ages if any portion of the part projects above the top of the tube.

Unfortunately, toy latex balloons are exempt from this small parts safety test. Balloons are often given away at restaurants and other events and are arguably the lowest cost and most popular toys purchased to delight young children. Most parents are aware of the dangers from choking. Indeed, each balloon package is required to carry a warning notice in a specified type size as follows:

“! WARNING: CHOCKING HAZARD—Children under 8 years can choke or suffocate on uninflated or broken balloons. Adult supervision required. Keep uninflated balloons from children. Discard broken balloons at once.”

Making such balloons safer is therefore most desirable, especially if it can be accomplished at low or no cost to the consumer.

Usually the neck portion of inflated toy balloons is knotted in order to seal the balloon, and usually affords no apparent or separate safety protection by itself. The applicant has submitted a separate patent application for a closure and sealing device for toy balloons that would afford a limited degree of protection by the use of such a device.

Past efforts at making toy balloons safer appear as an adjunct of attaching balloons to inflating and sealing devices that are large enough to discourage ingestion because of their physical size. In general, closure and sealing devices such as valves, disks or other mechanical closure means. Examples of a valve would be the applicant's U.S. Pat. No. 5,496,203, Balloon Valve Assembly, (Murray). Other sealing devices would include a molded plastic disk marketed under the trade name SAFETITE® DISK by Premium Balloon Accessories, Inc. of Sharon Center, Ohio, U.S. Pat. No. D359,229 (Jules). Each of these devices passes the aforementioned safety test for all ages. It has been found that in many cases the balloon neck will remain attached to the sealing device and thereby be less likely to be ingested, however these devices are comparatively heavy, expensive and their use, it is believed, limited to less than ½ percent of balloon's sold.

There is therefore a need for very inexpensive methods that make toy balloons safe by rendering portions of a toy balloon bad, unpalatable, offensive tasting, repulsive tasting, obnoxious or repugnant tasting, if the balloon is used in a manner which might lead to ingestion of the balloon or parts thereof. Within this application, the above terms are to be considered interchangeable. It is intended that a general degree of revulsion be conveyed by the words respective position in the list from bad tasting to unacceptably repugnant. In addition, by definition reference to safety coating 53 shall include and be an equivalent of repulsive taste agent 53,

The inclusion or application of a repulsive taste agent may be incorporated during manufacture of an inflatable member such as an elastomeric toy balloon or as part of a postproduction process as for example, when imprinting the elastometrc toy balloon, or as a means to process and incorporate the repulsive taste agent to an existing inventory of untreated balloons.

For some applications, for example inflating a toy balloon by mouth, it may be desirable to render some portions of the toy balloon, like the balloon neck relatively free of an unpalatable taste, or less repulsive than other portions.

The exterior surface may be made offensive tasting in areas where the balloon has been imprinted by the inclusion of an offensive taste agent in the ink applied to the exterior by imprinting machines, as for example U.S. Pat. No. 4,478,142 Balloon Printing Machine with Silk-screen (Santorpneous) and U.S. Pat. No. 4,829,894 Balloon Printing Machine, (Gardner), using ink containing a offensive taste agent while the interior surface of the main body might be substantially coated during the balloon inflation stage, for example, in preparation for printing, with a repugnant taste agent. If the balloon were to burst and create small chards, each of the small chards would be unpalatable and therefore expelled from the mouth, thereby decreasing the likelihood of injury or death from chocking.

Limited safety protection would be afforded by having portions of the exterior contain the repulsive taste agent. Far greater protection would be afforded by applying the repulsive taste agent to the balloon interior alone or in combination with another treatment, as for example a product such as Hi-Float™, U.S. Pat. No. 4,634,395 entitled “Inflatable Elastomeric Balloons Having Increased Buoyant Lifetimes” (Burchett), which is introduced into the balloon interior to lengthen the flying time of helium filled balloons.

By leaving the exterior of the neck portion of the toy balloon, substantially free of the offensive taste agent a user could inflate the balloon by mouth without encountering a substantial amount of offensive taste.

A procedure to provide an added degree of safety and to isolate the user more completely from an unpalatable taste during inflation would be to employ a balloon support inflation aid (similar to FIG. 5) and passes the aforementioned safety test. An example, the applicant's Balloon Valve Assembly (Murray U.S. Pat. No. 5,496,203) incorporated herein by reference. A cost saving improvement to the Murray valve would be to omit the valve flap while retaining the balloon support features. An alternative would be a simple valve-less tube as shown in FIG. 5 having head portion to support the balloon neck during inflation and a stem portion to keep the balloon further from the users face and thereby keep the neck portion from being propelled into the users throat if the balloon should burst during inflation or the user accidentally inhaling. The oval head of the Murray Balloon Valve Assembly has been found particularly easy to use and effective in supporting the balloon. Without a valve, the inflation aid offers the added benefit of making the balloon neck easier to pinch during inflation process.

### SUMMARY OF THE INVENTION

In accordance with one aspect of the invention, there is provided a safe toy balloon including (a) an inflatable member having an exterior surface, an interior surface, a neck portion and a body portion; and (b) a safety coating formed on one or more of said surfaces of the inflatable member, the safety coating consisting essentially of repulsive taste agent so that a person or child placing a broken balloon or small part of the balloon in the mouth will cause an automatic rejection and expulsion of the tainted parts thereby making toy balloon use safer.

In accordance with another aspect of the invention, there is provided a method of producing a safer toy balloon including (a) providing a dipping assembly containing at least one toy balloon material supporting mandrel; (b) providing a bank of vats including at least one vat containing liquid toy balloon material; (c) coating the at least one toy balloon material supporting mandrel with a repulsive taste agent to achieve a repulsive taste coated mandrel; and (d) dipping the repulsive taste coated mandrel into the at least one vat containing liquid toy balloon material to form a toy balloon having a repulsive interior coating from the repulsive taste coated mandrel and thereby cause an automatic rejection and expulsion of the tainted parts should the user attempt to ingest, thereby making toy balloon use safer.

### BRIEF DESCRIPTION OF THE DRAWINGS

In the detailed description of the invention presented below, reference is made to the drawings, in which:

FIG. 1 is an illustration of various parts of a toy balloon;

FIG. 2a is a perspective view of mandrel forms used in balloon production according to the present invention;

FIG. 2b is a perspective representation of vats used in balloon production;

FIG. 2c is a sectional view of a mandrel and location of repulsive taste agent forming the internal surface of balloon according to the present invention;

FIG. 2d is a sectional view of a mandrel and location of repulsive taste agent forming the external surface of balloon according to the present invention;

FIG. 3 illustrates a safe toy balloon after forming on mandrel form in according to the present invention;

FIG. 4 is a perspective view of balloon neck portions; and

FIG. 5 illustrates a safe toy balloon installed on an inflation aid.

### DETAILED DESCRIPTION OF THE INVENTION

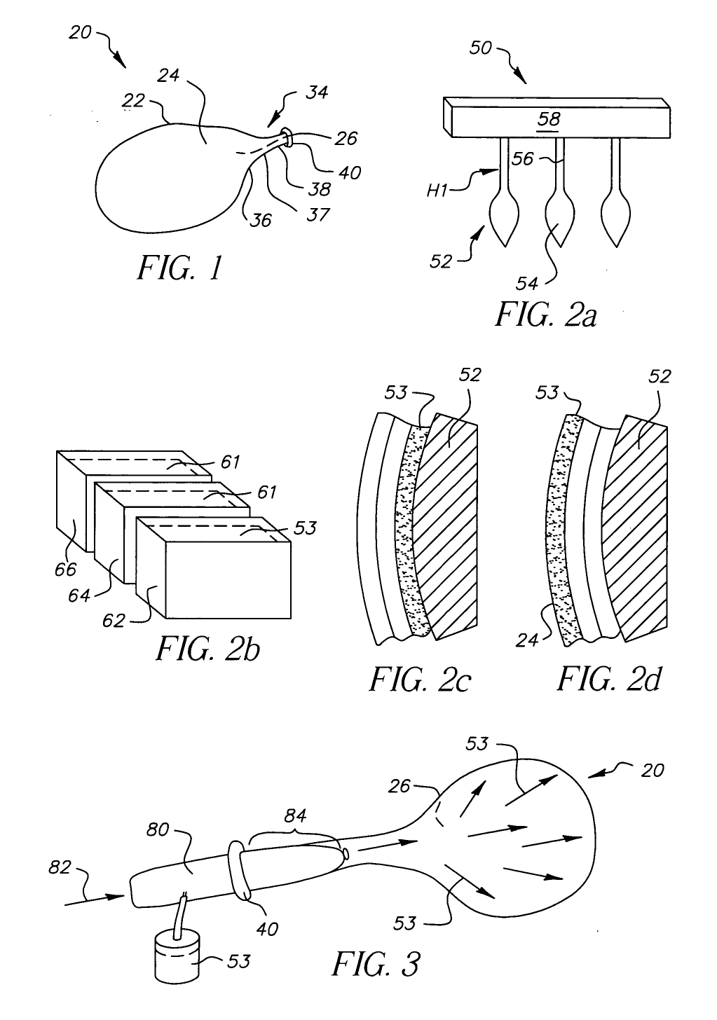

With reference now to FIGS. 1-5, reference numerals refer to the same or like elements.

FIG. 1 is an illustration for referencing various parts of an inflatable member such as a latex toy balloon or toy balloon 20. As shown, a toy balloon 20 includes a balloon body 22 having an exterior surface 24 and an interior surface 26. The balloon body 22 is contiguous with a balloon neck 34 consisting of neck portion 36, a mid neck portion 37, and near neck portion 38 adjoining a neck rim 40.

Referring now to FIGS. 2a-2d, FIG. 2a is a perspective view of mandrel forms used in balloon production according to the present invention showing a mandrel support 58 with a plurality of individual mandrel forms or mandrels 52, that together make up a dipping assembly 50 commonly used in balloon production. Individual mandrel forms 52 consist of mandrel body 54 and a mandrel neck portion 56. To produce a latex balloon, the dipping assembly 50 is dipped progressively into a series of vats 62, 64, 66 as shown in FIG. 2b comprising suitable balloon forming material such as suitable latex liquid material (not seen but well known), hereafter referred to simply as latex liquid 61, although it is usually a formulation of latex with other materials. The progressive dipping, similar to a candle making process, builds up the thickness of the balloon 20 until the desired thickness is obtained. This is illustrated in FIG. 2c, a sectional view of a mandrel and location of repulsive taste agent 53 forming the internal surface of balloon according to the present invention.

FIG. 2b is a perspective representation of vats used in balloon production. The first embodiment of the present invention consists of a first vat 62 containing a revolting or repulsive or obnoxious tasting agent 53 such as Denatonium Benzoate or other combinations of suitable materials. The remaining plurality of vats 64-66 contains untainted liquid latex 61. According to C Tech Corporation, Denatonium Benzoate is the world's bitterest known substance and is widely used to denature many products such as anti-freeze. In the preferred embodiment, (shown in FIG. 2c), a safety coating containing an repulsive or obnoxious tasting agent 53, as is applied to mandrel 52 by dipping into a vat 62 (shown in FIG. 2b) containing the repulsive or obnoxious tasting agent 53 to a depth H1 that, as shown, includes all of mandrel body 54 and about half of mandrel neck 56, thereby leaving the balloon neck portion free of the repulsive taste agent 53.

Alternately, the repulsive or obnoxious tasting coating 53 may be applied to height or depth H1 of the mandrel 52 by spray or other means for the purpose of coating the interior surface 26 of the toy balloon with safety coating 53. In this case, the first vat 62 therefore need not include the repulsive taste agent 53. A toy balloon formed after spraying or precoating the mandrel 52 as such will have an interior surface 26 that incorporates the repulsive taste agent 53 so that if broken after inflation, interior surfaces 26 of substantially all parts of the balloon will be tainted with the repulsive taste agent 53 and therefore likely to be expelled if a user should attempt to ingest it.

In another embodiment the entire interior surface 26 of toy balloon 20 including the interior of neck 34 contains the repulsive taste agent 53 and isolation of the repulsive taste be provided employing a balloon support device as will be described in FIG. 5.

FIG. 2d is a sectional view of a mandrel 52 and location of repulsive taste agent 53 on the exterior surface of the balloon. To produce this embodiment, balloon mandrel 52 is first dipped into a progression of liquid latex vats 66 and 64, and lastly into vat 62 containing the repulsive taste agent 53 thereby creating a safety coating on the external surface 24 of balloon 20, this having an advantage, for example, of not having the taste agent 53 build up on the mandrel or interfere with the balloon releasing there from. The option exists to evert balloon 20 so the safety coating would then be on the interior surface 26 and the exterior relatively devoid of the safety coating and unpalatable taste. The safety coating would perform its function if the balloon were to burst and thereby expose the repulsive taste agent.

Yet another method of safety coating the exterior surface of a toy balloon with a repulsive taste agent could be accomplished by bringing the toy balloons into contact with media containing the repulsive taste agent, for example tumbling with cloth wetted with a suitable carrier mixture, such as poly glycol and the repulsive taste agent. A dry powder mixture could optionally be employed. Again, the option would exist to evert the toy balloons in a manner common in the manufacture of latex gloves,

FIG. 3 shows yet another embodiment of the invention, an repulsive or unpleasant taste agent 53 introduced into the fluid stream 82, by means similar to an engine carburetor atomatizing of fuel, used to inflate the toy balloon and thereby safety coat and render the interior surface 26 unpleasant tasting. In this case, the entire interior surface of the toy balloon will have the repulsive taste, except for an interior portion 84 of balloon neck 34, seen in FIG. 3, is in contact with fluid filling nozzle 80 and thus protected from receiving the safety coating and associated repugnant taste.

FIG. 3 is a perspective view of fluid filling nozzle 80 adapted to introduce safety coating 53 to the interior surface 26 of toy balloon 20. Safety coating 53 may be a fluid or powder introduced into fluid stream represented by arrow 82 by any suitable means as suggested diagrammatically by FIG. 3.

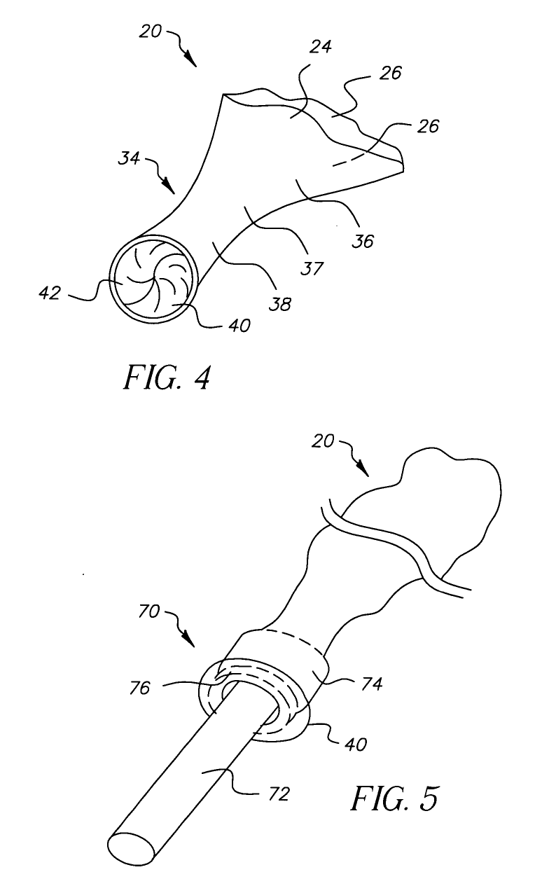

FIG. 4 is a perspective view of balloon neck 34, having portions 36, 37, 38 and neck rim 40, which surrounds orifice 42, and serves to illustrate still another embodiment of the invention. The introduction of media carrying the revolting or repulsive tasting agent 53 into the balloon interior through neck opening 42 for the purpose of coating the interior surface 26 of toy balloon 20. The media, for example might be an ingestible gel, powder, or liquid, like polypropylene glycol mixture. A user might introduce the media and then spread it over the inside surface 26 by massaging balloon body 22 of the balloon. By controlling the penetration and positioning of the media a greater portion of coating will be applied to the body portion than the neck portion 34 so that inflation by mouth will present minimal revolting taste to the user. Such media may be introduced prior to the customer receiving the balloon.

FIG. 5 illustrates a toy balloon removably installed on an inflation aid 70 for isolating the repulsive or unpleasant taste of the balloon interior during inflation by mouth of the toy balloon 20. The applicant's “Balloon Valve Assembly”, U.S. Pat. No. 5,496,203 (Murray) would serve such a purpose well. While helpful, the inflation aid 70 need not contain a valve. The aid consists of a stem 72, an oval head 74 for supporting toy balloon 20 and a stepped portion 76, which supports balloon neck rim 40 and has been found to aid significantly in securing a tight seal between the balloon 20 and inflation aid 70 during inflation. Such an aid, increases the safety of balloon use during inflation by lessening the risk of balloon neck ingestion should the balloon explode and propel the balloon neck into the users mouth, or the user accidentally inhale during inflation. Following inflation the user may twist the balloon neck and tie a knot the customary fashion or close and seal the balloon by using a device such as the applicant's Related Application, (Applicant's Docket MSD01), entitled “A SAFE TOY BALLOON CLOSURE AND SEALING DEVICE AND ASSEMBLIES USING SAME”.

As can be seen, there has been provided a method of producing a safe toy balloon 20 including comprising: (a) an inflatable member or toy balloon 20 having an exterior surface 24, an interior surface 26, a neck portion 34 and a body portion 22; and (b) a safety coating 53 formed on one or more of the surfaces of the inflatable member, the safety coating consisting essentially of repulsive taste agent.

Also there has been provided a method of producing a safe toy balloon 20 including (a) providing a dipping assembly 50 containing at least one toy balloon material supporting mandrel 52; (b) providing a bank of vats, 62-66, including at least one vat containing liquid toy balloon material 61; (c) coating the at least one toy balloon material supporting mandrel 52 with a repulsive taste agent 53 to achieve a repulsive taste coated mandrel; and (d) dipping the repulsive taste coated mandrel into the at least one vat containing liquid toy balloon material 61 to form a toy balloon having a repulsive interior coating from the repulsive taste coated mandrel.

In accordance with another aspect of the invention, there is provided a safe toy balloon including (a) an inflatable member or toy balloon 20 having an exterior surface 24, an interior surface 26, a neck portion 34 and a body portion 22; and (b) a safety coating 53 formed on selected surfaces of inflatable member or toy balloon 20, the safety coating consisting essentially of repulsive taste agent.

To further recap, means were shown for both making and for post processing a toy balloon 20 to incorporate and associate a safety coating 53 containing a repulsive taste agent with the exterior surface 24 of toy balloon 20, or portions thereof, and then reversing the toy-balloon thereby causing the repulsive taste agent 53 become associated with the interior surface of the toy balloon.

It was further shown that a safety coating 53 could be optionally be associated with both the interior surface 26 as well as the exterior surface 24, or portions thereof, of toy balloon 20 in the same or in differing degrees of repulsiveness of the safety coating 53.

While preferred embodiments of the invention have been illustrated and described, and while other alterations will be apparent, it should be understood that other variations would become apparent to one skilled in the art without departing from the principals herein. Accordingly, the invention is not to be limited to the exact configurations illustrated in the drawings.

What is claimed is:

1. A safe toy balloon comprising:
(a) an inflatable elastomeric member having an exterior surface, an interior surface, a neck portion and a body portion; and
(b) a safety coating formed on one or more of said surfaces of said inflatable member, said safety coating consisting essentially of repulsive taste agent.

2. The safe toy balloon of claim 1, wherein said safety coating is formed on only a designated portion of said at least one of said exterior surface and said interior surface of said inflatable member.

3. The safe toy balloon of claim 1, wherein said repulsive taste agent comprises a bittering agent.

4. The safe toy balloon of claim 2, wherein said designated portion of said inflatable member comprises a part of said body portion.

5. The safe toy balloon of claim 2, wherein said designated portion is not co-extensive with said neck portion of said inflatable member.

6. The safe toy balloon of claim 3, wherein said bittering agent comprises Denatonium Benzoate.

7. A method of producing a safe toy balloon comprising:
(a) providing a dipping assembly containing at least one toy balloon forming material supporting mandrel;
(b) providing a series of vats including at least one vat containing liquid toy balloon forming material;
(c) dipping said at least one toy balloon forming material supporting mandrel into said at least one vat containing liquid toy balloon forming material to form a toy balloon on said at least one toy balloon forming material supporting mandrel; and
(c) coating said toy balloon so formed with a repulsive taste agent resulting in a safe toy balloon having a repulsive tasting coating that will cause a person placing a piece of such toy balloon in the mouth to automatically and immediately reject and expel such piece, thereby making such toy balloon safe to use.

8. The method of claim 7, wherein coating said toy balloon so formed with a repulsive taste agent is achieved by coating said at least one balloon forming material supporting mandrel with the repulsive taste agent.

8. The method of claim 8, wherein said series of vats includes a first vat in said series containing said repulsive taste agent.

9. The method of claim 8, wherein said series of vats includes a last vat in said series containing said repulsive taste agent.

10. The method of claim 7, wherein said repulsive taste agent comprises a bittering agent.

11. The method of claim 10, wherein said bittering agent comprises Denatonium Benzoate.

12. The method of claim 8, wherein said balloon forming material supporting mandrel includes a balloon body supporting portion and a balloon neck supporting portion, and wherein coating said toy balloon so formed with a repulsive taste agent is achieved by coating only said balloon body supporting portion with said repulsive taste agent.

13. The method of claim 7, wherein coating said toy balloon so formed with a repulsive taste agent comprises spraying said repulsive taste agent onto said balloon material supporting mandrel.

14. The safe toy balloon of claim 7, wherein coating said toy balloon so formed with a repulsive taste agent comprises printing on said toy balloon using a printing process.

15. A method of producing a safe toy balloon comprising:
(a) providing a dipping assembly containing at least one toy balloon forming material supporting mandrel;
(b) providing a series of vats including at least one vat containing liquid toy balloon forming material;
(c) dipping said balloon forming material supporting mandrel into said at least one vat containing liquid toy balloon forming material to form a toy balloon; and
(d) coating an interior of said formed toy balloon by introducing a repulsive taste agent into said interior of said formed toy balloon.

16. The method of claim 15, wherein introducing said repulsive taste agent comprises blowing said repulsive agent with a fluid into an interior of said formed toy balloon.

17. A method of producing a safe toy balloon comprising:
(a) providing a dipping assembly containing at least one toy balloon forming material supporting mandrel;
(b) providing a series of vats including at least one vat containing liquid toy balloon forming material;
(c) dipping said balloon forming material supporting mandrel into said at least one vat containing liquid toy balloon forming material to form a toy balloon;
(d) coating the exterior of said formed toy balloon by applying a repulsive taste agent onto said exterior of said formed toy balloon; and
(e) reversing surfaces of said toy balloon such that said exterior surface becomes an interior surface of said formed toy balloon.

18. The method of claim 171 wherein coating said exterior surface comprises dipping said balloon forming material support mandrel into a last vat containing a repulsive taste agent.

19. The method of claim 17, wherein coating said exterior surface comprises spraying said repulsive taste agent onto said exterior surface.

20. The method of claim 17, wherein said bittering agent comprises Denatonium Benzoate.
